(19) United States
(12) Patent Application Publication
(10) Pub. No.: US 2024/0216764 A1
(43) Pub. Date: Jul. 4, 2024

(54) A TESTING DEVICE FOR A TENNIS BALL

(71) Applicant: Richard James WALKER, Hurstpierpoint, Hassocks (GB)

(72) Inventor: Richard James WALKER, Hurstpierpoint, Hassocks (GB)

(21) Appl. No.: 18/554,419

(22) PCT Filed: Mar. 28, 2022
(86) PCT No.: PCT/EP2022/058170

§ 371 Date: Oct. 6, 2023

(30) Foreign Application Priority Data
Apr. 8, 2021 (GB) 2104993.7

(51) Int. Cl. A63B 47/00; A63B 102/02; G01B 5/00; G01L 17/00

(52) U.S. Cl. CPC A63B 47/008; G01B 5/0023; G01L 17/005; A63B 2102/02; A63B 2220/56

(57) ABSTRACT

The invention provides a device for testing internal pressure of a hollow sports ball; the device comprising:
(i) a main body (2);
(ii) a removable cap (4); the main body (2) and cap (4) each being provided with mutually engaging formations (12, 14) that enable the main body (2) and cap (4) to be removably secured together by a twisting movement;
(iii) a load cell (46) and associated electronic circuitry for processing an output from the load cell (46); and
(iv) an electronic display (41) linked to the electronic circuitry for displaying an indicator of the internal pressure of the ball;
the main body having an interior void which, together with the removable cap, forms an enclosed ball-receiving chamber for receiving the ball;
the interior void of the main body being provided at an inner end thereof with a seat (44) on which the ball can sit, the load cell (46) being disposed beneath the seat (44); and the associated electronic circuitry optionally being disposed below the pressure sensor;
whereby the ball-receiving chamber is configured such that when the ball is in place in the chamber, the securing together of the main body and cap brings the main body and cap together in an axial direction to compress the ball and apply a pressure through the ball to the load cell, and a force signal sent from the load cell to the associated electronic circuitry is processed to enable the indicator of the internal pressure of the ball to be displayed.

This invention relates to a device for testing a parameter indicative of the internal pressure of a ball, particularly a tennis ball.

### BACKGROUND OF THE INVENTION

Tennis balls are formed from two rubber hemispherical shells bonded together and covered with two approximately figure-of-eight shaped pieces of felt. The tennis balls can either be pressurised (by the injection of compressed air or compressed nitrogen) or unpressurised.

Appendix I of the International Tennis Federation (ITF) Rules of Tennis (2021 edition) sets out the requirements that tennis balls must meet in order to be used in competitions. Appendix I specifies three main types of tennis ball for use in competitions, namely Type 1 (Fast), Type 2 (Medium) and Type 3 (Slow). In addition, there is a High Altitude ball type for use on courts above 1,219 metres (4000 feet) above sea level, and there are various types of ball for use in competitions where the competitors are ten and under years of age.

Type 1 (Fast) balls are specified for use on slow pace (Category 1) court surfaces, i.e. court surfaces which have an ITF Court Pace Rating of 0 to 29. This category includes clay courts and other types of unbound mineral surface.

Type 2 (Medium) balls are specified for use on medium-slow (Category 2), medium (Category 3) and medium-fast (Category 4) pace court surfaces which have ITF Court Pace Ratings of 30 to 34 (medium slow pace), 35 to 39 (medium pace) or 40 to 44 (medium fast pace). These categories include most acrylic coated surfaces plus some carpet surfaces.

Type 3 (Fast) balls are specified for use on fast (Category 5) pace surfaces which have an ITF Court Pace Rating of 45 or more. This category includes most natural grass, artificial grass and some carpet surfaces

According to Annex I of the ITF Rules, Type 1, Type 2 and Type 3 balls must all have a weight in the range from 56.0-59.4 grams (1.975-2.095 ounces). Type 1 and Type 2 balls must have a size within the range 6.54-6.86 cm (2.57-2.70 inches) whereas Type 3 balls must have a size in the range 7.00-7.30 cm (2.76-2.87 inches).

The extent to which the balls must bounce (rebound) is also specified in Appendix I. Thus, the rebound range for Type 1 balls is 138-151 cm (54-60 inches) whereas, for Type 2 and Type 3 balls, the required rebound range is 135-147 cm (53-58 inches).

The internal pressure of a tennis ball is major factor in determining how the tennis ball will bounce. Pressurised balls will progressively lose pressure after use and consequently will not bounce (rebound) to the same extent as they did when new.

For major tournaments, new balls will be used and hence the loss of internal pressure is not an issue. For smaller competitions, e.g. club tournaments and competitions between clubs, and for social and practice play, it is not practical or economical to use new balls each time but it is nevertheless desirable to know whether a ball is suitable for a given type of court surface. Thus, there is a need for a way of testing used tennis balls to check whether their internal pressures (and hence the rebound range) are at an acceptable level for use on a given court surface.

A number of devices have been disclosed for use in testing the internal pressures of tennis balls.

For example, U.S. Pat. Nos. 6,360,613 and 6,612,183 (both in the name of Iggulden) each disclose a container for storing tennis balls which incorporates a device for testing the playing condition of the ball. The container has a removable cap which is provided with a pressure indicator. In use, the cap is removed from the container, a ball is placed within the cap, and the cap is then attached to the closed end of the container so as to compress the ball between the cap and the external surface of the closed end of the container, enabling an indication of the internal pressure of the ball to be obtained. Although it is suggested that a spring-operated indicator or an electronic display coupled to a pressure transducer can be used, a preferred embodiment is disclosed as being a non-electrical liquid pressure indicator of the type described in U.S. Pat. No. 3,987,699.

U.S. Pat. No. 6,612,183 further discloses a mechanically-operating pressure testing device comprising a disc and a displaceable indicator arm, the displacement of which under compression by a tennis ball is indicative of the internal pressure of the ball.

U.S. Pat. No. 5,760,312 (Mackay) discloses a pressurised ball-testing device comprising a housing having two half shells, the interiors of which together define a ball-receiving chamber. A ball such as a tennis ball is placed inside the ball-receiving chamber and the two half shells are pushed together against the restoring force of the ball and a spring in the lower part of the device to urge protrusions into the ball. The extent of displacement of the surface of the ball, or the force needed to bring about a given displacement are then converted into a ball pressure indicator. A disadvantage of this device is that it requires calibration by the user, which is based on an assumption that the user will have a ball suitable for use as a standard in the calibration procedure. A further potential disadvantage of the Mckay device is that the compression and force-measuring mechanism includes a pre-compressed spring, with the consequent possibility that the spring may become weaker over time and hence the accuracy of the device will diminish.

U.S. Pat. No. 5,760,312 (Reenstra) discloses a device for measuring the compressibility of tennis balls. The device comprises a chamber with a hinged lid into which a tennis ball is placed. A force sensor, which is preferably a force sensing resistor, is located at the bottom of the chamber. As the lid is closed and secured by means of a latch, it compresses the tennis ball and the force transmitted through the tennis ball is sensed by the force sensing resistor. The force sensed by the sensor is converted into an output to a pair of LED indicators, and optionally an audio output, indicative of the state of compressibility of the ball. It is considered that a device as disclosed in U.S. Pat. No. 5,760,312 will suffer from a number of disadvantages. Firstly, the present applicant has found that force sensing resistors are not sufficiently robust for prolonged use and will therefore lose accuracy over a relatively short period of time. Secondly, because of the relatively long distance along the lid between the hinge and the latch, it is likely that the lid, even though reinforced, will bend and deform with use making it more difficult to close the lid and leading to a reduction in the compressive force of the lid and hence a reduction in accuracy of the device.

Further examples of tennis ball testers are disclosed in UK patents numbers GB230250A and GB328407A, German patent application DE 19710340A1 and U.S. Pat. No. 5,760,312.

### SUMMARY OF THE INVENTION

The present invention provides an improved device for testing the internal pressures of balls and in particular tennis balls.

Accordingly, in a first aspect of the invention, there is provided a device for testing internal pressure of a hollow sports ball; the device comprising:
(i) a main body;
(ii) a removable cap; the main body and cap each being provided with mutually engaging formations that enable the main body and cap to be removably secured together by a twisting movement;
(iii) a pressure sensor and associated electronic circuitry for processing an output from the load cell; and
(iv) an electronic display linked to the electronic circuitry for displaying an indicator of the internal pressure of the ball;
   the main body having an interior void which, together with the removable cap, forms an enclosed ball-receiving chamber for receiving the ball;
   the interior void of the main body being provided at an inner end thereof with a seat on which the ball can sit, the pressure sensor being disposed beneath the seat; and the associated electronic circuitry being optionally disposed below the pressure sensor;
   whereby the ball-receiving chamber is configured such that when the ball is in place in the chamber, the securing together of the main body and cap brings the main body and cap together in an axial direction to compress the ball and apply a pressure through the ball to the pressure sensor, and a force signal sent from the pressure sensor to the associated electronic circuitry is processed to enable the indicator of the internal pressure of the ball to be displayed.

The pressure sensor is preferably a load cell.

The main body is typically provided with a lower compartment, located beneath the interior void, the lower compartment containing the electronic circuitry.

The main body may comprise an upper main body section and a base section, the upper main body and base section being configured so that, when secured together, they form the said lower compartment.

The main body and cap are each provided with mutually engaging formations that enable the main body and cap to be removably secured together by a twisting movement, the twisting movement resulting in the cap and main body being moved together in an axial direction to compress the ball in the ball-receiving chamber.

By “twisting movement” is meant that the securing of the cap to the main body involves at least some relative rotation of the cap and/or main body to draw the cap and main body together or move them apart.

In one embodiment, the said formations take the form of complementary threads on the cap and main body that allow the cap and body to be screwed together.

In another embodiment, the cap and main body are provided with mutually engaging formations that cooperate to form a bayonet fitting between the cap and body.

In each case, the formations on the cap and main body are configured so as to limit the extent of movement together of the main body and cap so that a consistent degree of compression can be applied to the ball.

In one particular embodiment, the mutually engaging formations are formations that cooperate to form a bayonet fitting between the cap and body. Thus, for example, the main body may have in an upper end of a wall thereof one or more bayonet fitting slots and the cap may be provided with one or more lugs that engage the one or more bayonet fitting slots. Alternatively, the cap may be provided with the one or more bayonet fitting slots and the main body may be provided with the one or more lugs.

The main body may have a cylindrical or substantially cylindrical outer surface. Thus, the main body is typically substantially cylindrical.

The interior void of the main body is typically substantially cylindrical.

The cap is typically substantially cylindrical, and is usually of a shallow cylindrical shape: i.e. the diameter of the cylindrical shape is greater than the depth (length) of the cylinder.

Preferably, each of the cap, main body and the interior void of the main body are substantially cylindrical.

Thus, the ball-receiving chamber is preferably cylindrical.

The greater part of the volume of the ball-receiving chamber (typically more than 50%) is defined by a void in the main body of the device. More usually, at least 60% of the volume of the ball-receiving chamber is defined by a void in the main body of the device. Preferably, at least 75% of the volume of the ball-receiving chamber is defined by a void in the main body of the device.

The device is typically compact in shape and size. For example, it may have a maximum height of 150 mm and a maximum width (e.g. diameter) of 90 mm. In one embodiment intended for testing tennis balls, the device has a maximum height of 115 mm and a maximum width (e.g. diameter) of 80 mm.

The seat is typically concave or comprises an array of ridges or protrusions that are functionally equivalent to a concave surface. By “functionally equivalent to a concave surface” is meant that the crests or tips of the ridges or protrusions lie approximately on a three dimensional curve which is concave.

The concave seat or the array of ridges and protrusions are shaped so as to conform substantially to part of the spherical surface of the ball, for example an area of up to 30% of the spherical surface of the ball.

In one embodiment, the seat is concave and has a radius of curvature corresponding generally to that of a tennis ball.

In another embodiment, the seat comprises one or more annular ridges in which the ball can sit.

The cap may be provided with a centring formation for centring the ball.

The centring formation may comprise a single annular ridge or a plurality of concentric spaced apart annular ridges on an undersurface of the cap.

In one embodiment, the centring formation comprises a plurality of concentric spaced apart annular ridges which decrease in depth from a radially innermost ridge to a radially outermost ridge.

In another embodiment, the centring formation comprises a plurality of concentric spaced apart annular ridges which increase in depth from a radially innermost ridge to a radially outermost ridge.

In another embodiment, the centring formation may comprise a concave (in the direction of the ball) surface or an array of ridges and protrusions that are functionally equivalent to a concave surface.

In another embodiment, the centring formation may comprise a plurality (e.g. two or three) of concentric ridges or ribs that increase in depth in a radial outwards direction such that each ridge or rib can contact the ball.

In another embodiment, the centring formation may comprise a plurality (e.g. two or three) of discontinuous concentric ridges or ribs that increase in depth in a radial outwards direction such that each ridge or rib can contact the ball.

It has been found that a device provided with a concave or functionally concave seat and/or a centring formation provides more consistent and accurate pressure readings than a device provided with flat surfaces for compressing a tennis ball. It has been found that when the compression surfaces are flat (for example as in the device disclosed in U.S. Pat. No. 5,760,312), pressure readings will often fluctuate depending on the orientation of the tennis ball inside the ball-receiving chamber; i.e. whether the orientation is such that contact between the compression surface and the ball is on the seam of the tennis ball or away from the seam. This is thought to be because a flat compression surface will make a point contact with the tennis ball and, where the point contact is made with a relatively recessed part of the tennis ball surface (i.e. the seams), the pressure reading will differ from when the point contact is with an area of the tennis ball surface away from the seam. The concave or functionally concave seat and/or a centring formation contact the ball over a much larger area and hence the seam has little or no effect on the pressure reading. Accordingly, accurate measurements can generally be made regardless of the orientation of the ball.

In an alternative embodiment, the cap may have an underside which is provided with a ball-compressing formation comprising a convex (in the direction of the ball) surface or an array of ridges and protrusions that are functionally equivalent to a convex surface. The benefit of the convex surface or array is that it provides a smaller initial contact area with the ball and therefore less effort is required from the user in fastening the lid down on the ball.

In another embodiment, the cap may have an underside which is provided with a plurality (e.g. two or three) of concentric ridges or ribs that decrease in depth from an innermost ridge outwards. In this embodiment, the innermost ridge serves the twin functions of centring the ball whilst providing a smaller initial contact area with the ball so as to reduce the effort required from the user in fastening the lid down on the ball.

In one embodiment, the ball-compressing formation may comprise a plurality (e.g. two or three) of discontinuous concentric ridges or ribs that decrease in depth from an innermost ridge outwards. In this embodiment also, the innermost ridge serves the twin functions of centring the ball whilst providing a smaller initial contact area with the ball so as to reduce the effort required from the user in fastening the lid down on the ball

The load cell is disposed beneath the seat, so that movement of the seat when the ball is compressed results in a pressure being applied to the load cell.

The seat in which the ball can sit is typically mounted on or forms part of a flexible floor of the ball-receiving chamber, i.e. a flexible floor of a void in the main body.

It is preferred that the device is capable of testing the internal pressure of more than one type of ball.

In one embodiment, the device is provided with a selector switch which is linked to the electronic circuitry so that a user of the device can select from one of plurality of different types of ball to be tested. For example, the device may be set up so that the user can selected one of a plurality (e.g. two three of four) of types of tennis ball to be tested.

By way of example, the device can have three different tennis ball settings to allow the testing of Type 1, Type 2 and Type 3 balls as specified in the ITF Rules of Tennis.

The switch can one which comprises a mechanical actuator such as a button, rotating knob, or sliding knob, or it can be an electronic switch such as a region of a touch screen.

In another embodiment, the electronic circuitry comprises or is linked to a receiver (e.g. a Bluetooth receiver) for receiving an electronic signal from an external device (such as a Bluetooth transmitter in a mobile phone or tablet computer) so that an instruction can be received to select one of plurality of different types of ball to be tested. For example, a device such as a mobile phone or tablet computer can be used to scan a bar code or product code on a container containing tennis balls and a signal identifying the type of tennis ball sent to the receiver in the device of the invention. Alternatively, or additionally, the external device can be used to manually identify (e.g. by manual input of the type of ball, or by selecting from a series of options offered by the external device) and the identity of the type of ball to be tested transmitted to the device of the invention. In one embodiment, a mobile phone or other portable electronic communications device such as a tablet computer can be provided with an App that enable it to be used together with a pressure testing device of the invention.

The device of the invention is provided with an electronic display linked to the electronic circuitry for displaying an indicator of the internal pressure of the ball. The electronic display may be a screen which displays alphanumeric information indicative of the internal pressure of the ball. For example, the screen may display the absolute internal pressure of the ball. Alternatively, or additionally, the screen can display a message, index figure or letter or other visual indicator as to whether the internal pressure is within an acceptable range for a given purpose (e,g. competition on a particular type of court surface).

In an alternative, or additionally, the electronic display may comprise an array of one or more lights (e.g. LED lights), preferably a plurality of lights, which light up to provide an indication of the internal pressure of the ball. For example, the electronic display may comprise an LED strip having at least five LEDs, more usually from about six to about twenty LEDs (for example from eight to twelve LEDs), wherein the electronic circuitry is configured to provide an extent of illumination of the LED strip indicative of a particular internal pressure state of the ball and/or suitability for use on a given type of surface. In one embodiment, the LED strip has twelve individual LEDs. The LED strip is typically covered by a protective diffuser layer or cover formed from, for example, a transparent or translucent (preferably translucent) plastics material.

The electronic display, electronic circuitry and the load cell (or other pressure sensor) typically form part of the main body. More particularly, the electronic circuitry and load cell (or other pressure sensor) can be contained within a lower part of the main body portion and the electronic display (e.g. LED strip) can be mounted in or on a wall of the lower part of the main body portion.

The device is preferably battery powered, although it may be provided with a connector and/or transformer for use with mains electricity. In a preferred embodiment, the device is provided with a rechargeable battery and a connector for connection to a charging point. For example, the device may be provided with a USB port (e.g. a USB-C) port for connection to a power supply.

In one embodiment, the device is provided with a tilt switch so that the device can be turned on simply by tilting or shaking the device. Where a tilt switch is provided, the electronic circuitry can be programmed to turn the device off if it is not used within a certain period of time, for example after 30-60 seconds.

In another aspect, the invention provides a device for testing internal pressure of a ball; the device comprising a cylindrical main body, a cylindrical removable cap, a pressure sensor, associated electronic circuitry for processing an output from the pressure sensor, and an electronic display linked to the electronic circuitry for displaying an internal pressure parameter indicative of the internal pressure of the ball; an interior void of the main body and the removable cap together forming a cylindrical ball-receiving chamber for receiving the ball; wherein the chamber is provided with a concave seat in which the ball can sit, the pressure sensor being associated with the concave seat; and the cap is provided with a ball-centring formation on a downwardly depending inner surface thereof; the cap and main body being provided with interlocking formations that allow the cap and main body to be moved together in an axial direction to compress the ball and hold the cap and main body in a ball-compressing position, at which position a pressure applied to the pressure sensor by the ball when compressed is converted by the associated electronic circuitry to the internal pressure parameter which is displayed by the electronic display.

In this aspect of the invention, the pressure sensor is preferably a load cell.

The pressure sensor (e.g. load cell) is typically disposed beneath the seat, so that movement of the seat when the ball is compressed results in a pressure being applied to the load cell.

The interlocking formations preferably form a threaded connection or bayonet fitting.

In one preferred embodiment, the interlocking formations form a bayonet fitting.

It will be appreciated from the foregoing description that the invention provides a device which is readily portable and can be used quickly and easily to test whether the internal pressure of a ball, particularly a tennis ball, has the correct internal pressure, and hence degree of bounce, for it to be used on a given type of surface. Thus, the user simply switches on the device, disconnects the cap from the main body, inserts the ball into the ball-receiving chamber and then secures the cap on the main body, e.g. by screwing or twisting (in the case of a bayonet fitting) it on, so that the ball is compressed between the cap and a pressure sensor in the main body. The pressure parameter can then be read off from the electronic display and an assessment made as to whether it is within a required range for a given ball type. It will be appreciated that, where the device is provided with a selector switch which allows a user to select from one of plurality of different types of ball to be tested, the user is able to check whether the correct ball type has been selected for the surface on which they are playing and, if necessary, is able to adjust the setting to the correct ball type, before carrying out the test.

### BRIEF DESCRIPTION OF THE DRAWINGS

FIG. 1 is a view from one side of a pressure testing device according to one embodiment of the invention.

FIG. 2 is a view from above of the device shown in FIG. 1.

FIG. 3 is a view from below of the device shown in FIG. 1.

FIG. 4 is a sectional elevation along line A-A in FIG. 1.

FIG. 5 is an exploded view of the device shown in FIG. 1.

FIG. 6 is an exploded view from direction D1 of the device shown in FIG. 5.

FIG. 7 is a perspective exploded view of the device shown in FIG. 1.

FIG. 8 is a sectional elevation along line B-B in FIG. 5.

FIG. 9 is a view from one side of a pressure testing device according to a second embodiment of the invention.

FIG. 10 is a view from above of the device shown in FIG. 9.

FIG. 11 is a view from below of the device shown in FIG. 9.

FIG. 12 is a sectional elevation along line A-A in FIG. 9.

FIG. 13 is an exploded view of the device shown in FIG. 9.

FIG. 14 is an exploded view from direction D2 of the device shown in FIG. 13.

FIG. 15 is a perspective exploded view of the device shown in FIG. 9.

FIG. 16 is a sectional elevation corresponding to the exploded view shown in FIG. 14.

FIG. 17 shows an example of display graphics on the display screen in the embodiment of FIGS. 9 to 17.

### DETAILED DESCRIPTION OF THE INVENTION

The invention will now be illustrated in more detail (but not limited) by reference to the specific embodiments shown in the accompanying drawings.

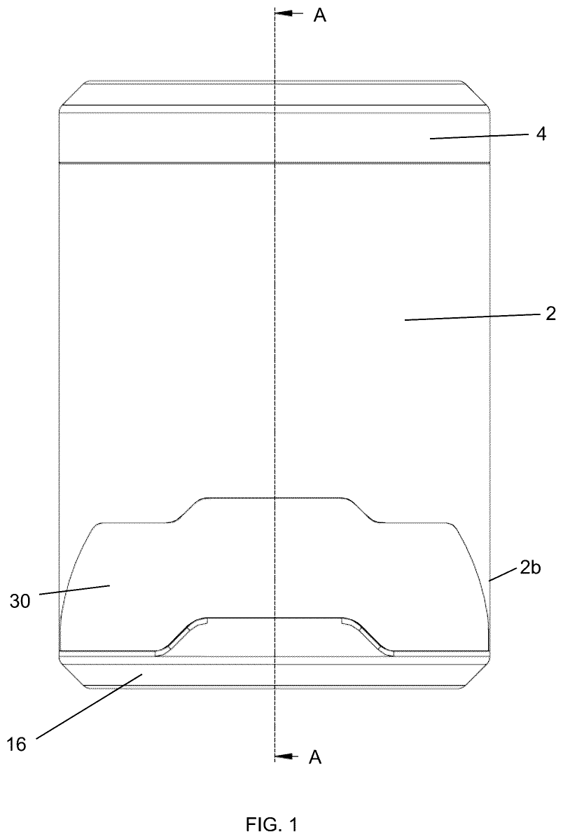

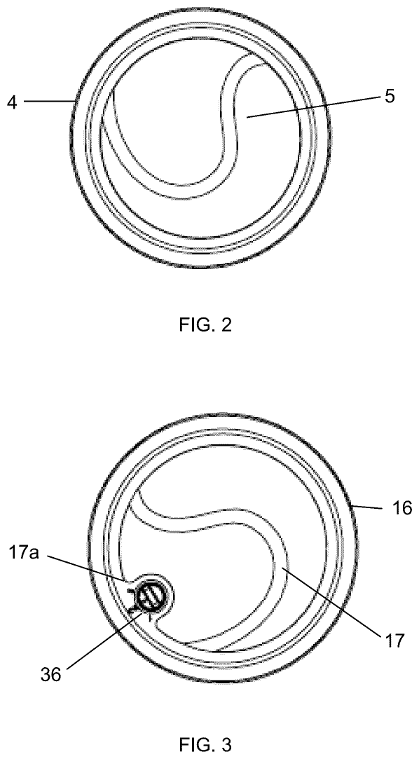

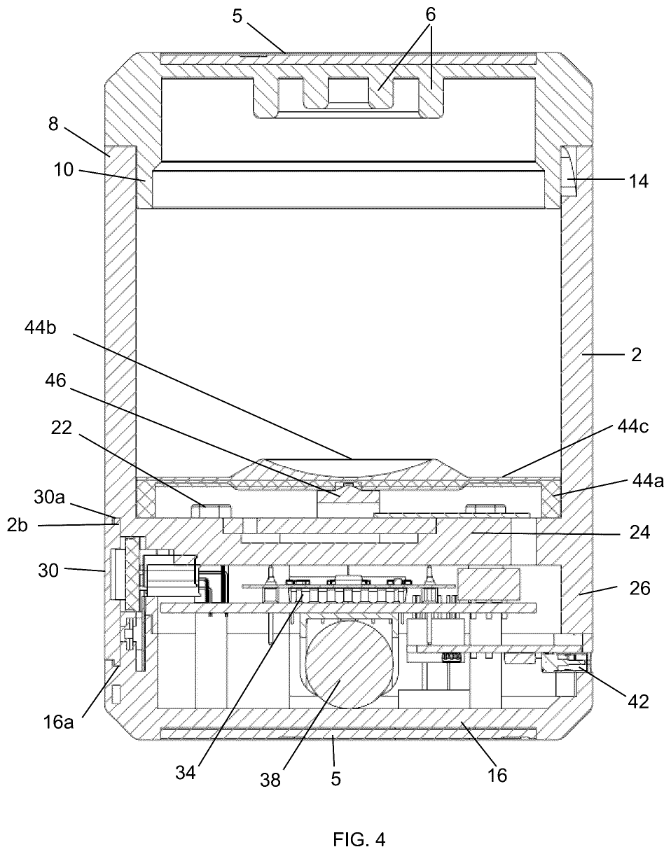

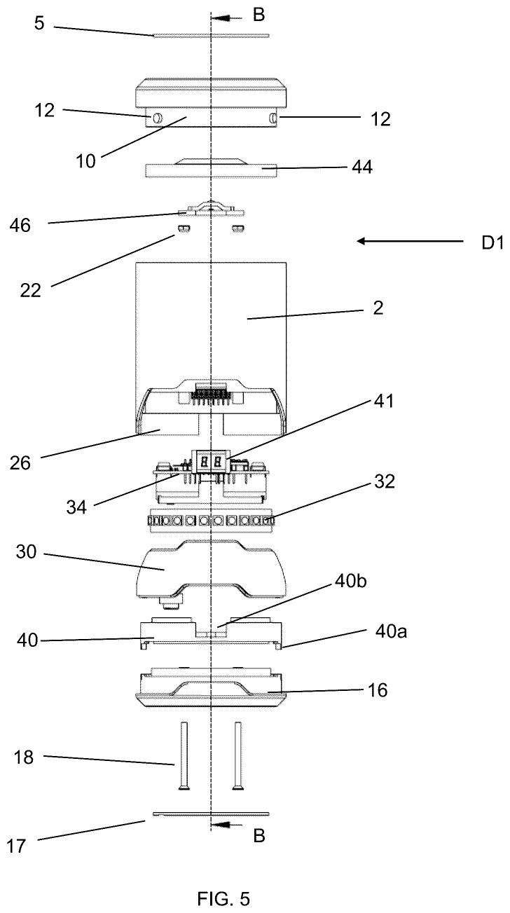

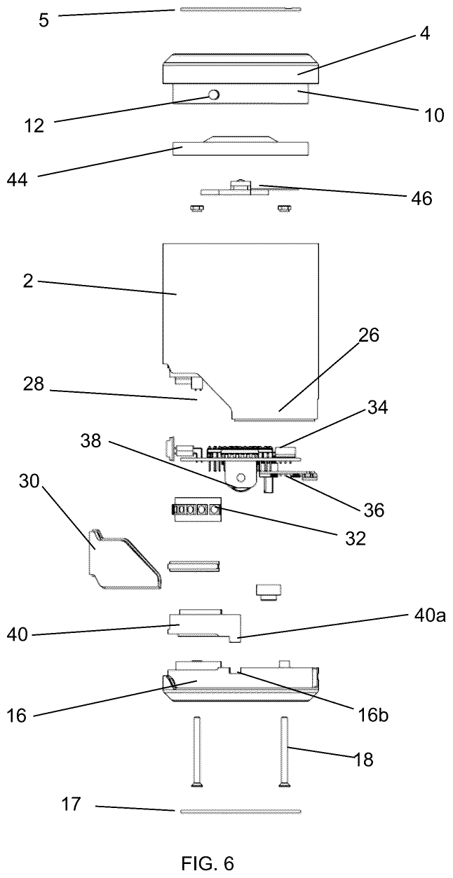

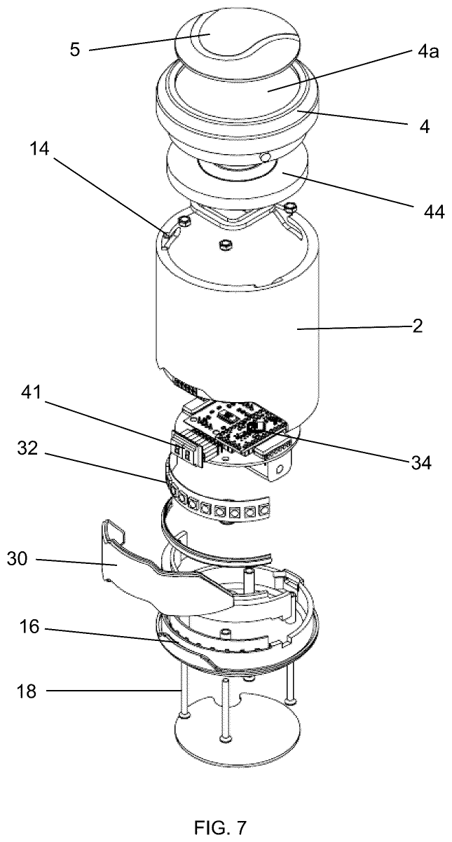

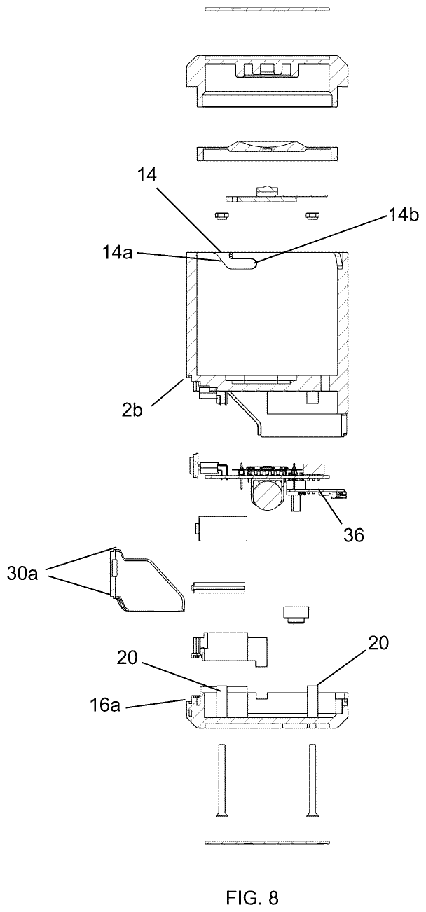

The device of FIGS. 1 to 8 is generally cylindrical in form and comprises a generally cylindrical main body (2) and a cap (4). The cap and main body can be formed from a metal or plastics material or combinations of metal and plastics materials. In one embodiment, the cap and main body are each formed from moulded plastics materials. A cover plate (5) bearing an impression of the seam on a tennis ball is set into a recess (4a) in an upper surface of the cap (4). Instead of depicting a tennis ball, the cover plate could instead bear a company logo or other advertising material.

The cap (4) has a pair of concentric downwardly oriented circular ribs (6) which are shaped to receive and hold an upper surface of a tennis ball. The cap has a downwardly oriented spigot portion (10) which fits inside the upper rim (8) of the main body (2). The spigot portion (10) has three radially outwardly projecting lugs (12) which fit into and engage open ended slots (14) in the wall of the main body portion (2). The open ended slots (14) each have an inclined ramp surface (14a) at the open end and a horizontal region (14b) at the closed end. The lugs (12) and slots (14) together make up a bayonet fitting.

At its lower end, the main body (2) is secured to a base (16) by means of screws (18) which extend through channel formations (20) in the base (16) and engage nuts or other threaded elements (22) mounted in or on a floor (24) of the main body portion. As an alternative to being a separate component, the base (16) can be integrally formed with the main body (2). The base has a circular recess on its underside and a lower cover plate (17) is set into the recess. The lower cover plate (17), which covers the fastening screws (18), bears a design creating the impression of a seam on a tennis ball. It will be appreciated however that alternative designs could be carried by the cover plate (17) or it can be left blank.

The main body (2) has a skirt portion (26) which extends below the floor (24) and has a cut-away or opening (28) on one side. The cut-away or opening (28) is filled by a translucent panel (30) which is held firmly between the main body (2) and the base (16) by means of a peripheral tongue (30a) around the edge of the panel (30) being retained in correspondingly shaped grooves (16a, 2b) in the base (16) and wall of the main body (2) when the main body (2) and base (16) are secured together by screws (18).

The space between the main body portion (2), translucent panel (30) and the base (16) contains an LED display strip (32) (for example a WS2812B RGB LED strip), electronic control circuitry on an integrated circuit board (34) (for example a Micro-IC ATmega328PB), a tilt switch (not shown), and a rechargeable battery (38). The tilt switch can be a switch-ball type SW-520D switch which is mounted on the circuit board (34). The rechargeable battery can be a 3.7V 14500 lithium ion battery controlled by an IC TC4056 battery management module.

A generally semi-circular wall element (40) rests on an upper edge of the base (16) and is held in place by means of tabs (40a) which engage correspondingly shaped recesses (16b) in a upstanding circumferential wall of the base (16). The semi-circular wall element (40) has a cut-out (40b) which accommodates a digital display (41) (e.g. a green 2-digit 7-segment LED display) and electrical connections between the integrated circuit board (34) and the digital display (41).

The LED lights of the LED display strip (32) and the digital display (41) are protected by virtue of being behind the translucent panel (30) but the LED lights of the display strip (32) and digits displayed on the digital display (41) can be seen through the panel.

A USB-C port (42) is set into the wall on one side of the base (16). The USB-C port is connected to the battery management system and the rechargeable battery and can be connected to a power source to allow recharging of the battery.

A 3-position rotatable switch (36) is set into the underside of the base (16) and can be accessed through a cut-away (17a) in the lower cover plate (17). The switch (36), which is connected to the integrated circuit board (34) (connection not shown), can be switched between three positions to allow the user to select the type of ball to be tested, for example:
1. Type 1 (designed for use on slow surfaces, such as clay);
2. Type 2 (designed for use on standard-speed surfaces, such as hard courts); and
3. Type 3 (designed for use on faster surfaces, such as grass).

On the upper side of the floor (24) of the main body is mounted a flexible seating element (44) comprising a cylindrical spacer wall (44a), a central seat formation (44b) and a flexible connecting wall (44c) linking the central seat formation (44b) and the spacer wall (44a). The seating element (44) is typically moulded from a resilient plastics material and/or rubber. The central seat formation (44b) has a part spherical surface of a radius of curvature corresponding generally to that of a ball (e.g. tennis ball) to be tested. A pressure sensor in the form of a load cell (46) is located in a space beneath the central seat formation (44b). The load cell is electrically connected (connection not shown) to the integrated circuit board (34). The load cell can be, for example, a 50 kg 3 wire type 925119 load cell.

In use, a user tilts the device to switch it on and can then inspect the setting of the switch (43) to see whether the type of tennis ball selected is the appropriate type for the surface on which they will be playing. If necessary or desired, the setting can be changed to a different ball type. Alternatively, if the user usually plays on a particular type of surface and usually uses a particular type of ball, the initial step of inspecting the switch setting can be omitted.

Having inspected and optionally changed the switch setting, or having decided simply to use the previous setting, the cap of the device is then removed by twisting it clockwise to release it from the bayonet fitting and a ball is placed inside the main body so that the lower surface of the ball sits in the central seat formation (44b).

Once a ball has been placed inside the main body portion, the spigot portion (8) of the cap is inserted into the upper rim (8) of the main body portion (2) so that the lugs (12) are aligned with the open ends of the slots (14). The cap (4) is then pushed downwards against the restoring force of the tennis ball and twisted so that the lugs (12) move down the ramped surface (14a) and into the horizontal region (14b) of the slot (14). This has the effect of bringing the cap (4) and main body (2) together and compressing the ball. The force applied to the ball by compression between the cap (4) and main body (2) is transmitted through the ball and the seat (44b) of the flexible seating element (44) on to the load cell (46). The magnitude of the force applied by the compressed ball through to the load cell will depend on the internal pressure within the ball. A ball which has lost some of its internal pressure will impart a smaller force to the load cell than a ball which still has its original internal pressure.

The load cell is connected to the electronic control circuitry and the signal from the load cell is converted into an internal pressure figure for the ball which is then displayed on the digital display (41). At the same time, the LED lights of the LED display strip (32) light up, the number of illuminated lights and the percentage of the display strip illuminated indicating the pressure status of the ball. Thus, for example, a new ball having its intended internal pressure will produce a display in which all of the LED lights are lit up, whereas a ball which has lost pressure will produce a display which have only a proportion of the LED lights illuminated. Thus, the user is able to see the actual internal pressure of the ball as well as an indication whether the pressure is within the requisite range for the ball to be used. The colours of the LED lights can also be used to indicate whether the ball is of an acceptable pressure. For example, the presence of illuminated green LED lights at one end of the LED strip can indicate an acceptable pressure whereas the presence of only regions showing red lights and no regions showing green lights can indicate insufficient internal pressure.

An advantage of the presence of the annular ribs (6) and the concave curvature of the seat (44b) is that the ball is in contact with the compression surfaces of the device over a much larger area than would be the case if the compression surfaces were flat, and this has been found to avoid or substantially reduce inaccuracies that would otherwise arise because the presence of the recessed seams on the tennis ball.

Although the annular ribs and/or concave curvature of the seat help to even out the effect of any undulations in the surface of the tennis ball that result from the presence of the seams, and thereby significantly improve the accuracy of the device, it may also be advantageous to programme (i.e. pre-calibrate) the device so that it assumes that the ball is tested when in a particular orientation within the ball-receiving chamber, for example, with the logo on the ball facing upwards. In such a case, an instruction may be provided (either in written materials provided with the device or applied to the device as a sticker or label, or marked on the device, or indicated by the electronic display on the device, or any electronic device such as a smart phone or tablet device linked to the test device) to place the ball in the device in the preferred orientation. In this way, the accuracy and reproducibility of the test results may be even further enhanced.

A further advantage of the device of the invention is the speed with which the internal pressure of a ball can be checked. Thus, for example, it has been found that the pressure of a ball can be tested in around 4-5 seconds. Further advantages of the device include its lightness, compactness and portability. The minimal number of moving parts and the absence of mechanical elements such as springs means that the device should be highly reliable and durable. A still further advantage of the device is its versatility in enabling balls of different types (e.g. different types of tennis balls) to be tested using only a simple switch to change between ball types.

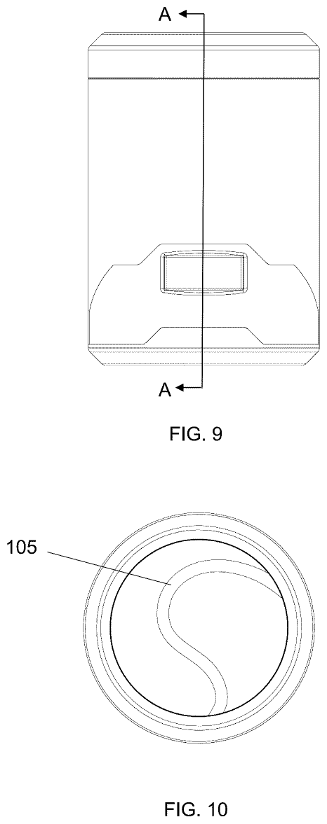

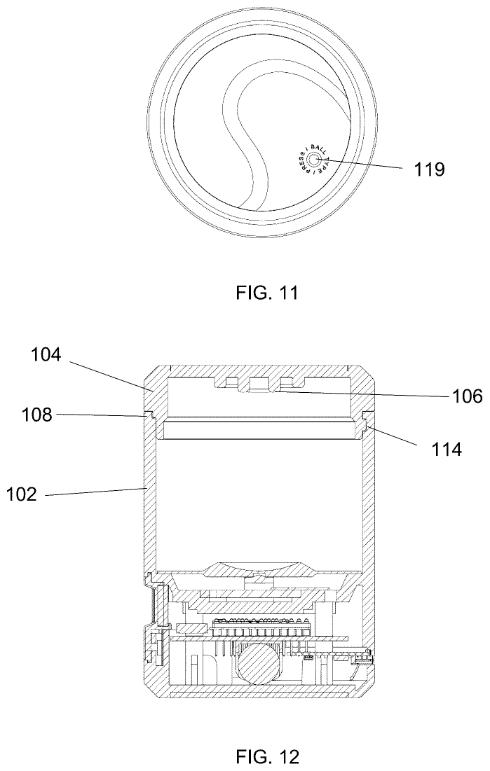

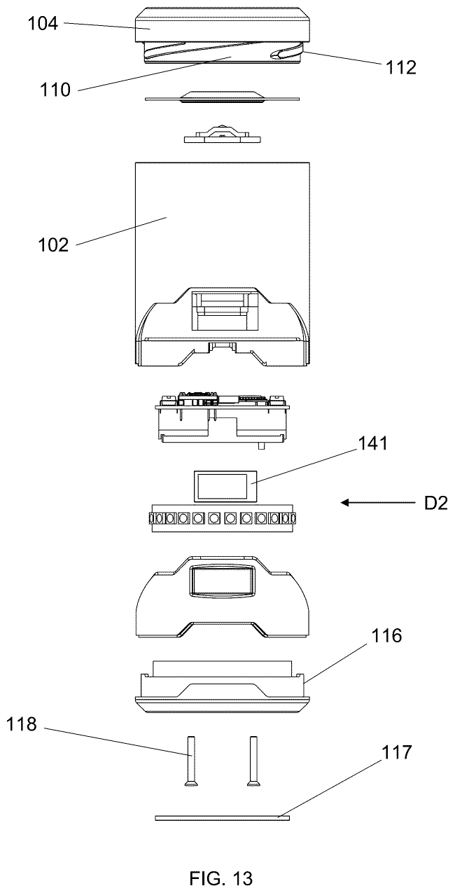

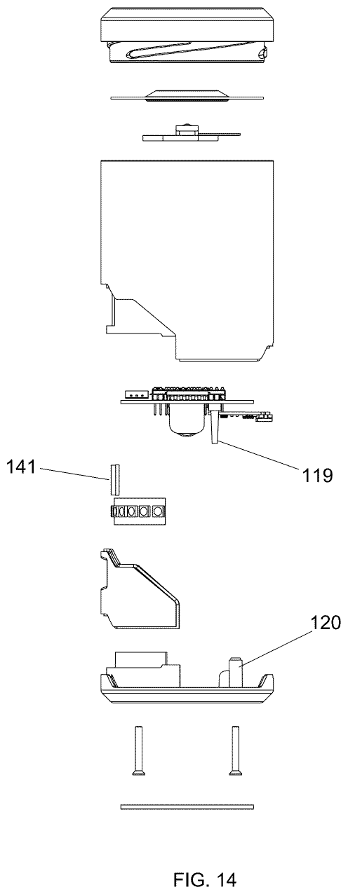

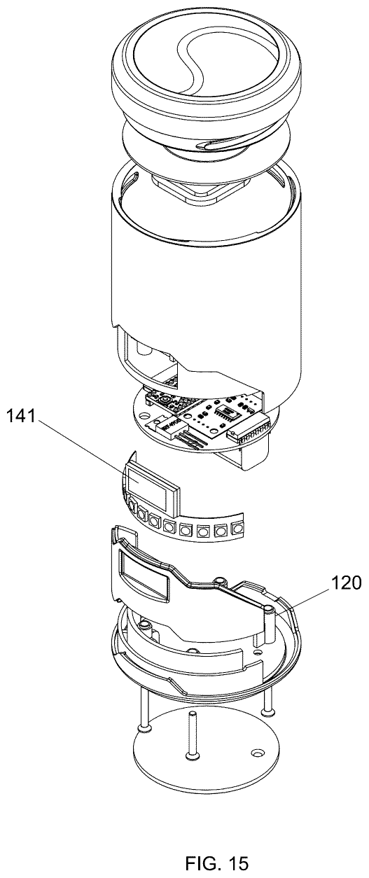

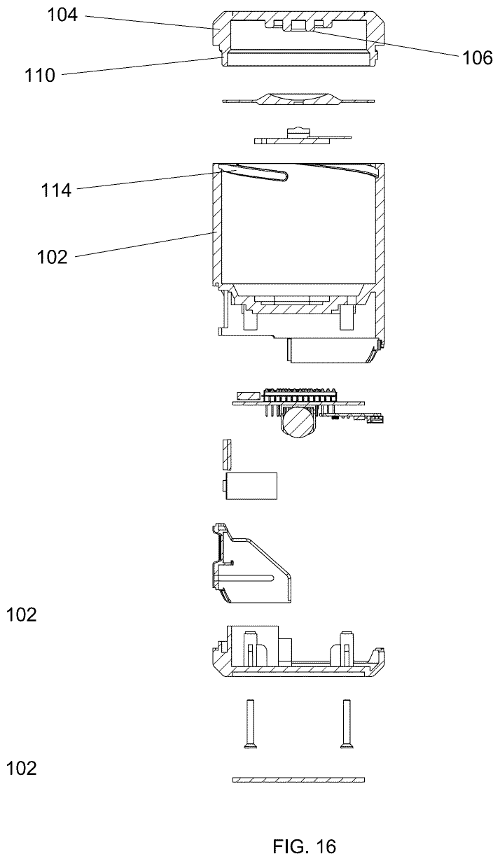

A second embodiment of the invention is shown in FIGS. 9 to 17. The features of the device of FIGS. 9 to 17 are largely the same as the device shown in FIGS. 1 to 8 but with several significant differences as described below. Thus, as with the embodiment of FIGS. 1 to 8, the embodiment shown in FIGS. 9 to 17 is generally cylindrical in form and comprises a generally cylindrical main body (102) and a cap (104). The upper surface of the cap bears an impression (105) of the seam on a tennis ball.

The cap (104) has a downwardly oriented spigot portion (110) which fits inside the upper rim (108) of the main body (2). The spigot portion (110) has a short external thread (112) which engages a corresponding thread (114) in the upper wall of the main body portion (102).

The undersurface of the cap (104) has a pair of concentric downwardly oriented circular ribs (106) which are positioned to engage an upper surface of a tennis ball when the cap (104) is screwed onto the main body (102). However, in contrast to the concentric ribs present in the embodiment of FIGS. 1 to 8, the concentric ribs are configured in a downwardly convex manner. This arrangement provides a smaller initial contact area with the ball, thereby increasing the pressure that the ribs will apply to the ball for the same amount of hand pressure applied by the user to the lid when fastening the lid down onto the ball. Therefore, less effort is required by the user in fastening the lid. The radially innermost rib also functions as a centring formation for the ball.

At its lower end, the main body (102) is secured to a base (116) by means of screws (118) which extend through channel formations (120) in the base (116) and engage nuts or other threaded elements (not shown) mounted in or on a floor of the main body portion. The base has a circular recess on its underside and a lower cover plate (117) is set into the recess. The lower cover plate (117), which covers the fastening screws (118), bears a design creating the impression of a seam on a tennis ball. Instead of the 3-position rotatable switch (36) in the underside of the base (16) of the device of FIGS. 1 to 8, the device of FIGS. 9 to 17 has concealed beneath the lower cover plate (117) a tactile push button switch (119) that can be activated by pressing the underside of the device to switch between different ball types.

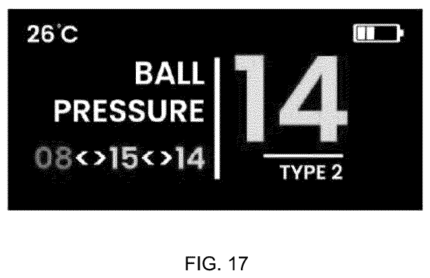

The interior components of the device of FIGS. 9 to 17 are largely the same as the device of FIGS. 1 to 8 but, instead of the digital display (41) of the device of FIGS. 1 to 8, the device shown in FIGS. 9 to 17 has a thin-film-transistor liquid-crystal colour display (TFT LCD) (141) which can, for example, be a 80×160 pixel display. As illustrated in FIG. 17, the TFT LCD can be used to display the ball pressure data for the current ball (large number 14) and several previously tested balls (small numbers 08< >15< >14), the type of ball (Type 2) and the battery status. The pressure data shown on the screen are typically colour coded to indicate whether the ball has the required pressure. For example, pressure figures in an acceptable range may be illuminated in a green colour whereas pressure figures that are not in an acceptable range may be illuminated in a red colour. Thus, in the embodiment shown in FIG. 17, the pressure figures of “14” and “15” shown on the screen are within an acceptable range and are consequently shown in a green colour whereas the pressure figure “08” is too low and therefore shows up in a red colour. A temperature sensor (not shown) within the device measures local temperatures and the measured temperature is also displayed on the screen.

The embodiments described above and illustrated in the accompanying figures are merely illustrative of the invention and are not intended to have any limiting effect. It will readily be apparent that numerous modifications and alterations may be made to the specific embodiments shown without departing from the principles underlying the invention. All such modifications and alterations are intended to be embraced by this application.

1. A device for testing internal pressure of a hollow sports ball; the device comprising:
(i) a main body;
(ii) a removable cap; the main body and cap each being provided with mutually engaging formations that enable the main body and cap to be removably secured together by a twisting movement;
(iii) a pressure sensor and associated electronic circuitry for processing an output from the pressure sensor; and
(iv) an electronic display linked to the electronic circuitry for displaying an indicator of the internal pressure of the ball;
the main body having an interior void which, together with the removable cap, forms an enclosed ball-receiving chamber for receiving the ball;
the interior void of the main body being provided at an inner end thereof with a seat on which the ball can sit, the pressure sensor being disposed beneath the seat; and the associated electronic circuitry optionally being disposed below the pressure sensor;
whereby the ball-receiving chamber is configured such that when the ball is in place in the chamber, the securing together of the main body and cap brings the main body and cap together in an axial direction to compress the ball and apply a pressure through the ball to the pressure sensor, and a force signal sent from the pressure sensor to the associated electronic circuitry is processed to enable the indicator of the internal pressure of the ball to be displayed.

2. A device according to claim 1 wherein the pressure sensor is a load cell.

3. A device according to claim 1 wherein the mutually engaging formations constitute complementary threads that allow the cap and body to be screwed together; or are formations that cooperate to form a bayonet fitting between the cap and body.

4. A device according to claim 3 wherein the mutually engaging formations are formations that cooperate to form a bayonet fitting between the cap and body.

5. A device according to claim 1 wherein the seat in which the ball can sit is mounted on or forms part of a flexible floor of the interior void of the main body.

6. A device according to claim 1 wherein the ball-receiving chamber is cylindrical.

7. A device according to claim 1 wherein the indicator of the internal pressure of the ball which is displayed by the electronic display is an alphanumerical value indicative of the internal pressure of the ball.

8. A device according to claim 1 wherein the display comprises an array of LED lights that light up in a manner indicative of the internal pressure of the ball and/or suitability for use on a given type of surface.

9. A device according to claim 1 having a first electronic display which displays an alphanumerical value indicative of the internal pressure, and a second electronic display in the form of an array of LED lights that light up in a manner indicative of the internal pressure of the ball.

10. A device according to claim 1 wherein a switch is provided to allow a user to select which of a plurality of types of ball is to be tested.

11. A device according to claim 1 wherein the seat at the inner end of the interior void of the main body is concave or comprises an array of ridges or protrusions that are functionally equivalent to a concave surface.

12. A device according to claim 11 wherein the concave seat or the array of ridges and protrusions are shaped so as to conform to part of the spherical surface of the ball, for example an area of up to 30% of the spherical surface of the ball.

13. A device according to claim 1 wherein the cap is provided with a centring formation for centring the ball.

14. A device according to claim 13 wherein the centring formation comprises a single annular ridge or a plurality of concentric spaced apart annular ridges on an undersurface of the cap.

15. A device according to claim 14 wherein the centring formation comprises a plurality of concentric spaced apart annular ridges which decrease in depth from a radially innermost ridge to a radially outermost ridge.

16. A device according to claim 1 which is provided with a tilt switch so that the device can be turned on by tilting or shaking the device

17. A device according to claim 1 wherein the electronic circuitry comprises or is linked to a receiver for receiving an electronic signal from an external device so that an instruction can be received to select one of plurality of different types of ball to be tested.

18. A device for testing internal pressure of a ball; the device comprising a cylindrical main body, a cylindrical removable cap, a pressure sensor, associated electronic circuitry for processing an output from the pressure sensor, and an electronic display linked to the electronic circuitry for displaying an internal pressure parameter indicative of the internal pressure of the ball; an interior void of the main body and the removable cap together forming a cylindrical ball-receiving chamber for receiving the ball; wherein the chamber is provided with a concave seat in which the ball can sit, the pressure sensor being associated with the concave seat; and the cap is provided with a ball-centring formation on a downwardly depending inner surface thereof; the cap and main body being provided with interlocking formations that allow the cap and main body to be moved together in an axial direction to compress the ball and hold the cap and main body in a ball-compressing position, at which position a pressure applied to the pressure sensor by the ball when compressed is converted by the associated electronic circuitry to the internal pressure parameter which is displayed by the electronic display.

19. A device according to claim 18 wherein the pressure sensor is a load cell.

20. A device according to claim 19 wherein the load cell is disposed beneath the seat.

21. (canceled)
